(19) United States
(12) Patent Application Publication
(10) Pub. No.: US 2017/0137824 A1
(43) Pub. Date: May 18, 2017

(54) NEW TREATMENT AGAINST INFLUENZA VIRUS

(71) Applicants: Indranil BANERJEE, Basel (CH); Ari HELENIUS, Zürich (CH); Patrick Daniel MATTHIAS, Basel (CH); Yasuyuki MIYAKE, Basel (CH); Yohei YAMAUCHI, Zürich (CH)

(72) Inventors: Indranil BANERJEE, Basel (CH); Ari HELENIUS, Zürich (CH); Patrick Daniel MATTHIAS, Basel (CH); Yasuyuki MIYAKE, Basel (CH); Yohei YAMAUCHI, Zürich (CH)

(21) Appl. No.: 15/318,638

(22) PCT Filed: Jun. 12, 2015
(86) PCT No.: PCT/IB2015/054460

§ 371 Date: Dec. 13, 2016

(30) Foreign Application Priority Data
Jun. 13, 2014 (EP) 14172323.9

(51) Int. Cl. C12N 15/113; C07K 16/40

(52) U.S. Cl. CPC C12N 15/1137; C07K 16/40; C12Y 305/01098; C07K 2317/569; C07K 2317/76; C12N 2310/14

(57) ABSTRACT

The present application provides a method for treating an influenza virus infection in a subject characterised in that a therapeutically effective amount of a modulator of the ubiquitin-binding property of HDAC6 is administered to said subject. The present application also provides an antibody binding to HDAC6 and decreasing or blocking its ubiquitin-binding properties for use in treating an influenza virus infection.

### FIELD OF THE INVENTION

The present invention provides a method for the treatment of influenza virus infections.

### BACKGROUND OF THE INVENTION

Influenza virus, especially Influenza A virus (IAV) is an enveloped virus of great medical impact; a highly contagious, constantly evolving, serious pathogen that causes seasonal epidemics and unpredictable, global pandemics with high mortality and morbidity in humans and other species. With the risk of an influenza pandemic growing, there is an important need to understand virus-host interactions in detail, and to develop new antiviral strategies (Fauci 2006).

IAV has a single-stranded, negative-sense RNA genome divided between 8 sub-genomic RNA molecules. These are individually packaged into helical ribonucleoproteins that contain the viral polymerase complex and multiple copies of a nucleoprotein (NP). In the mature virus particle, the set of eight vRNPs form a stable, capsid-like, supramacromolecular complex with the matrix protein (M1)(Noda and Kawaoka 2012). M1 forms a shell around the vRNPs and is peripherally associated with the inside surface of the viral envelope membrane. During IAV entry into a host cell, uncoating of the particle must occur; the envelope is lost and the network of interactions between the M1, the viral envelope, and the vRNPs are undone before the vRNPs are imported into the nucleus through nuclear pore complexes (Martin and Helenius 1991).

The capsid uncoating process is initiated in early endosomal vacuoles soon after receptor-mediated endocytosis of IAVs. Here, the virus is exposed to mildly acidic pH that triggers the opening of a channel protein, M2, in the viral envelope leading to influx of protons and a conformational change that renders the capsid uncoating-competent (Lamb, Zebedee et al. 1985; Zhirnov 1990; Martin and Helenius 1991; Pinto, Holsinger et al. 1992). When the pH in endosomal vacuoles drops to 5.5 or below, the membrane fusion function of the hemagglutinin (HA) is activated, and the capsid is transferred to the cytosol (Matlin, Reggio et al. 1981; White, Matlin et al. 1981). After penetration, M1 is released into the cytosol, and the vRNPs stripped of M1 are imported within half an hour into the nucleus for replication (Bukrinskaya, Vorkunova et al. 1982; Martin and Helenius 1991; Kemler, Whittaker et al. 1994; O'Neill, Jaskunas et al. 1995; Nevalainen, Nissinen et al. 2010; Banerjee, Yamauchi et al. 2013; Chou, Heaton et al. 2013).

### SUMMARY OF THE INVENTION

The present inventors found and show that in the absence of the deacetylase HDAC6, reduced virus titers are observed in vivo and in vitro. Moreover, the inventors found that whereas the deacetylase activity of HDAC6 is not important for the reduced titers, the ZnF-UBP domain of HDAC6, i.e. the ubiquitin-binding property of HDAC6, is required.

The present invention therefore provides a method for treating an influenza virus infection in a subject characterised in that a therapeutically effective amount of a modulator of the ubiquitin-binding property of HDAC6 is administered to said subject. In some embodiments, the modulator inhibits the ubiquitin-binding property of HDAC6. In some embodiments, the influenza virus is the influenza A virus. In some embodiments the modulator of HDAC6 decreases or silences the expression of HDAC6 and is, for example, a siRNA. In some embodiments, the modulator of HDAC6 is an antibody specifically binding to HDAC6, for instance a single-domain antibody.

The present invention also provides an antibody binding to HDAC6 and decreasing or blocking its ubiquitin-binding properties for use in treating an influenza virus infection. In some embodiments, this antibody is a single-domain antibody.

### DESCRIPTION OF THE FIGURES

FIG. 1: HDAC6 is required for capsid uncoating of IAV. (a) Schematic diagram showing the cellular entry steps of IAV. (b) Endocytosis assay: the endocytosed X31 particles were detected at 30 min post-internalization with an anti-HA antibody. Dynamin-dependent endocytosis was blocked with 80 μM dynasore (Dyn) 1 h prior to and during the assay. The cell membrane was stained with WGA-AF647. The number of endocytosed virus particles/cell was counted by Advanced Cell Classifier (ACC) program 14,45. (c) HA Acidification assay: after allowing virus internalization for 1 h, the acidified HA was stained with anti-A1 antibody that recognizes the acid-induced conformation of HA. Nuclei were stained with DRAQ5. To block endosomal acidification, the WT MEFs were treated with 50 nM bafilomycin A1 (BafA1). The fluorescence intensity of the A1 signal was measured by FACS analysis. (d) Fusion assay: the SP-DiOC18(3) and R18-labeled virus particles were allowed to enter the cells for 1.5 h. Fusion of the viral and vacuolar membranes was indicated by the appearance of dequenched DiOC18(3) signal. The fluorescence intensity of the fusion signal was detected by FACS analysis. (e) Uncoating assay: virus particles were allowed to enter the cells for 2.5 h. A mouse monoclonal antibody (HB64) was used to stain the viral M1. Actin filaments were stained with phalloidin-AF647. The fraction of cells with dispersed M1 signal was quantified by FACS analysis. Dispersion of M1 into the cytosol coincided with a dramatic increase in fluorescence intensity due to increased access of antibodies to M13. (f) Nuclear import assay: to detect the import of the incoming vRNPs into the nucleus, virus particles were allowed to enter the cells for 4 h. To stain NP, a mouse monoclonal antibody (HB65) was used. The number of nuclei positive for NP signal was quantified using the ACC program. The assays were performed in the presence of 1 mM cycloheximide to prevent synthesis of new viral proteins. Scale bars, 20 μm.

FIG. 2: HDAC6 facilitates capsid uncoating of IAV particles fused at the PM. (a) Schematic diagram showing the main steps of the acid-bypass uncoating assay. (b) Confocal images of acid-bypass uncoating assay in the WT (upper panel) and HDAC6 KO MEFs (lower panel). Following acid-bypass uncoating at pH 5.0 or pH 7.4 as a control, the cells were processed for IIF. The viral HA, M1 was detected using Pinda and HB64 antibody, respectively, and actin was stained with phalloidin-AF647. (c) Quantification of acid induced fusion at the PM of SP-DiOC18(3) and R18-labeled virus particles by FACS in WT and HDAC6 KO MEFs. (d) Quantification of acid-bypass uncoating assay by a MATLAB-based infection counter program to calculate the fraction of cells with dispersed M1 signal. Scale bars, 5 μm.

FIG. 3: The ubiquitin-binding domain of HDAC6 is crucial for capsid uncoating. (a) Schematic representation of (i) domain organization of HDAC6, and (ii, iii) its interaction with Ub chains with free C-terminal ends. HD: deacetylase domain, ZnF-UBP: zinc-finger ubiquitin binding domain. (b-e) Quantification of the uncoating, nuclear import, infection, and acid bypass uncoating assays using WT, HDAC6 KO, and HDAC6 rescue cell-lines HDAC6 (WTr), (HDm), and (ZnFm). (f) Western blot analyses of Ub, NP, and M1 following proteinase K treatment of purified X31 virions in presence/absence of Triton X-100. (g) Pull-down assay of purified, His-tagged HDAC6 full-length and deletion mutants after incubation with purified X31 lysates. Western blotting was done to detect HDAC6, His, Ub, C-terminal end of unanchored Ub, NP, and M1. (h) Immunoprecipitation of HDAC6 from cells infected with X31 for 2.5 h, using an anti-HDAC6 antibody. Western blotting was done to detect HDAC6, NP, M1, and PCNA.

FIG. 4: Reduced viral titers in the lungs of IAV-infected HDAC6 KO mice. WT and HDAC6 KO mice were infected intra-tracheally with 50 p.f.u. of IAV PR8. (a) Viral titers in the lungs were measured at day 3 and day 5 post-infection. (b) Differential cell composition of bronchoalveolar lavage (BAL)-infiltrating cells was analyzed at day 5 post-infection by FACS (alveolar macrophages (AM), inflammatory macrophages (iM), monocytes (MC) and neutrophils). (c) BAL-infiltrating CD4+, CD8+, and virus-specific NP34+CD8+ T cells were analyzed at day 10 post-infection. Bar graphs represent total cell numbers and the mean±SD (n=5). (d) Influenza PR8 HA-specific antibody concentrations of the IgA isotype in the BAL at day 10, determined by ELISA. All data are represented as mean±SD. ns: P>0.05, *P<0.05.

FIG. 5: IAV infection after Ab delivery. In a preliminary experiment, A549 cells were transfected with indicated antibodies using Ab Deliverin (OZ Biosciences) reagent, according to the manufacturer's instructions. 2 hours after transfection, after which 30% of the cells contained transfected antibodies, IAV X31 was super-infected for 5 hours, followed by fixation and staining for viral NP and FACS analysis. The results show the relative percentage of infection in cells transfected with anti-NP polyclonal (xx), anti-HDAC6 2ZnF antibodies, and anti-HDAC6 Ub-binding domain antibody, compared to a non-related antibody against the mitochondrial protein Tom120 (Santa Cruz).

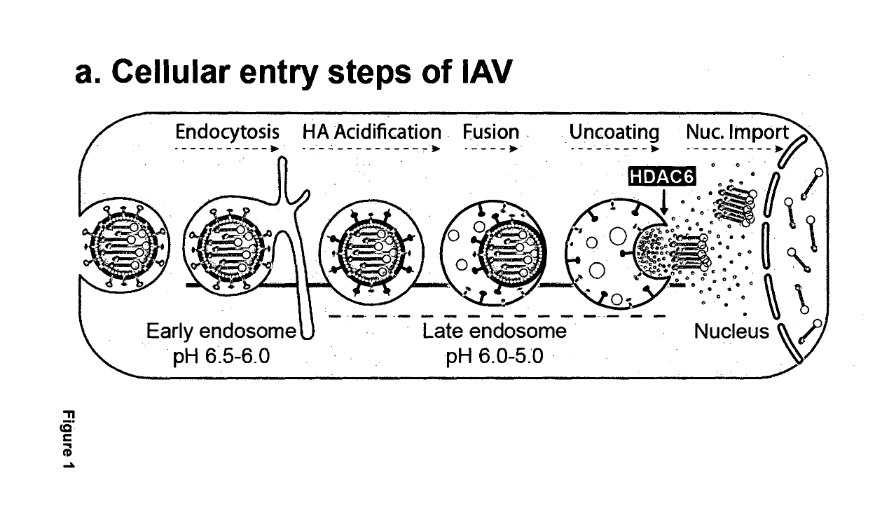

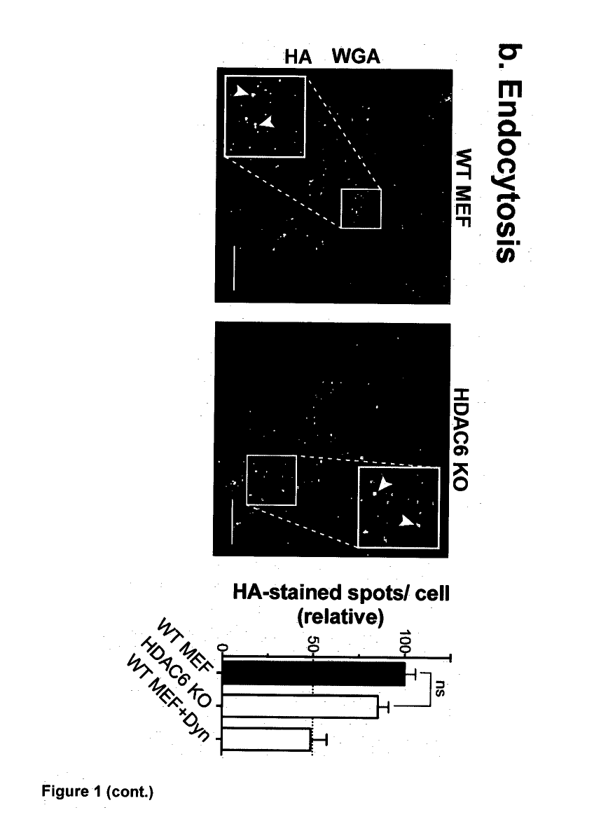

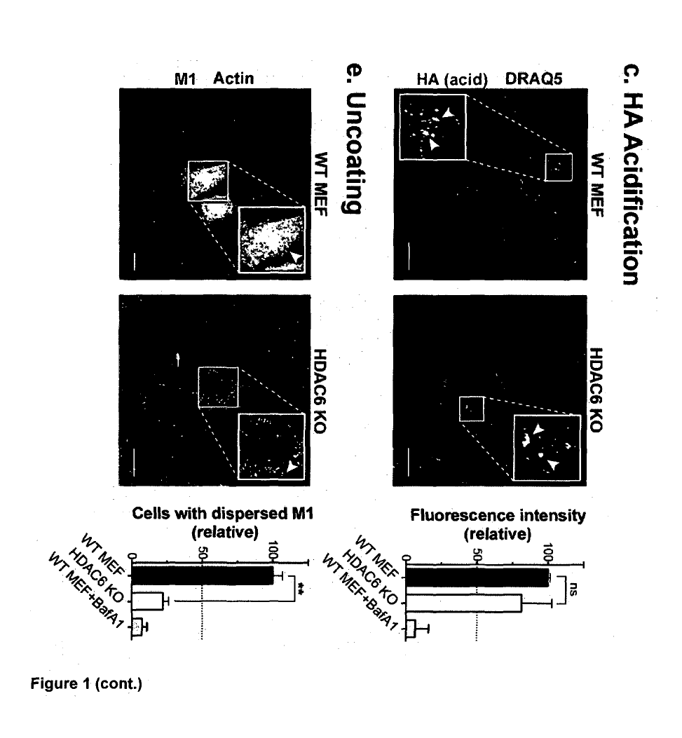

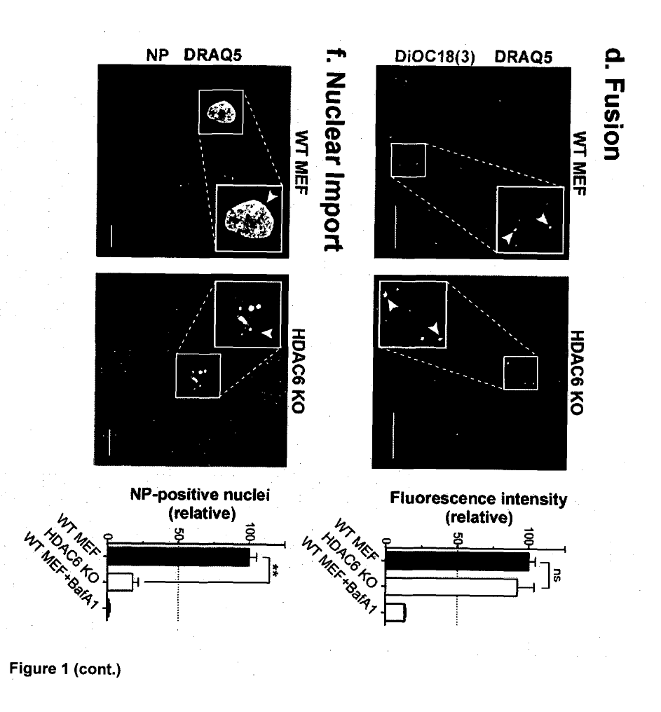

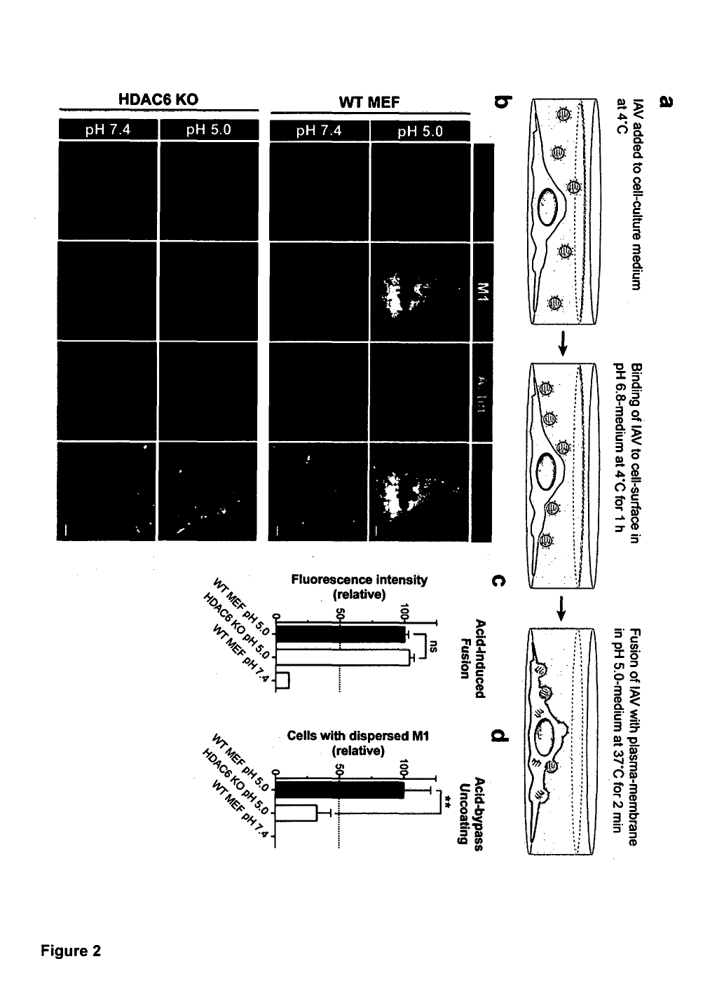

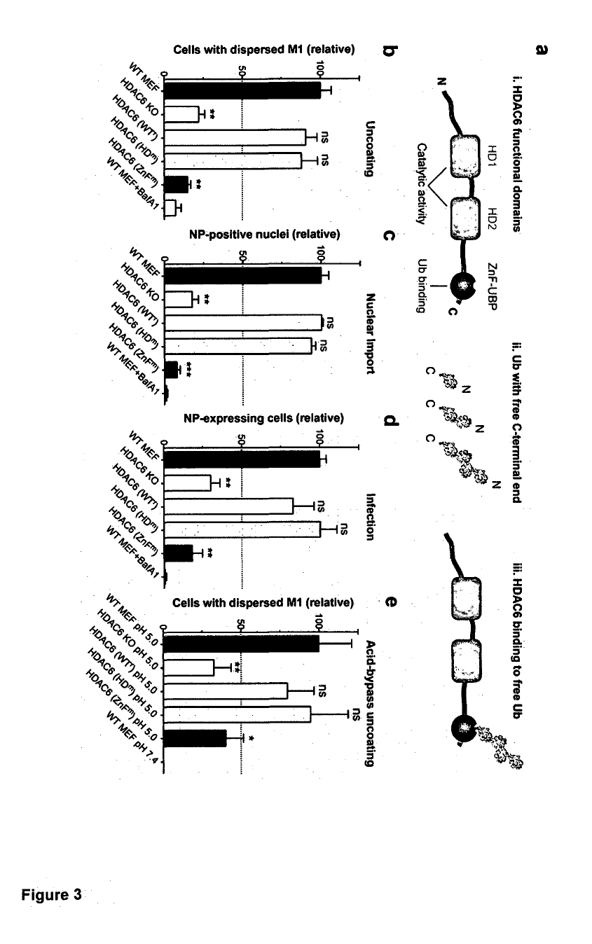

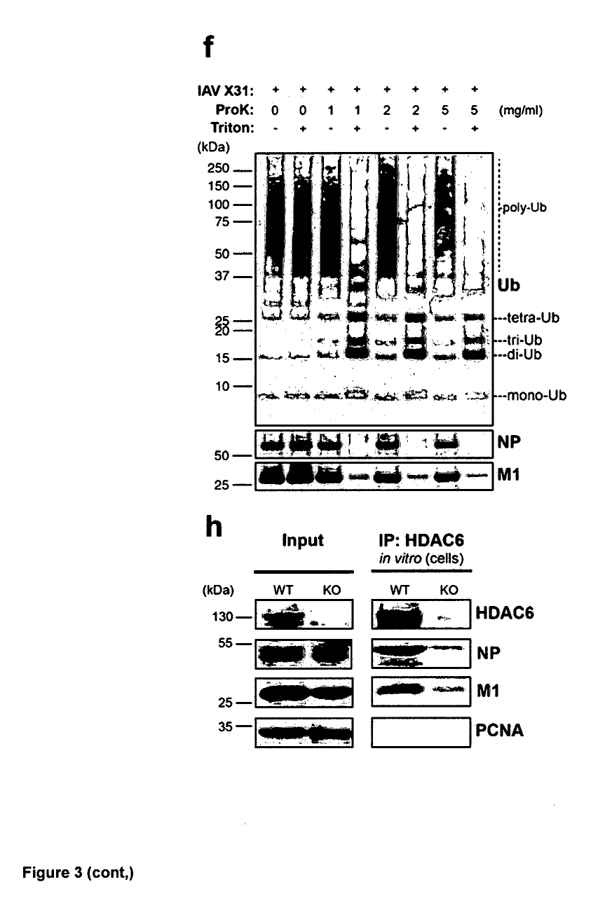

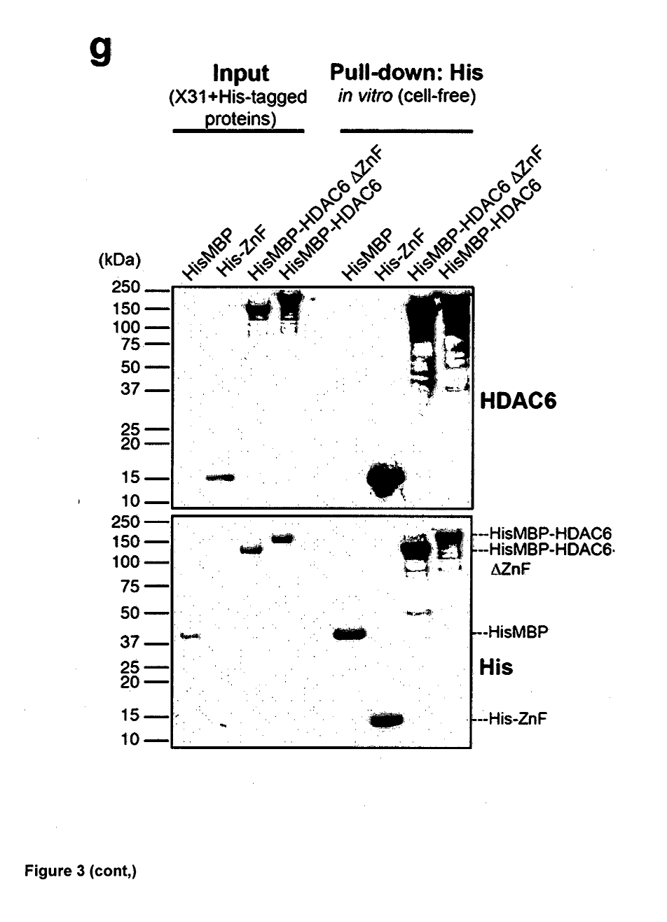

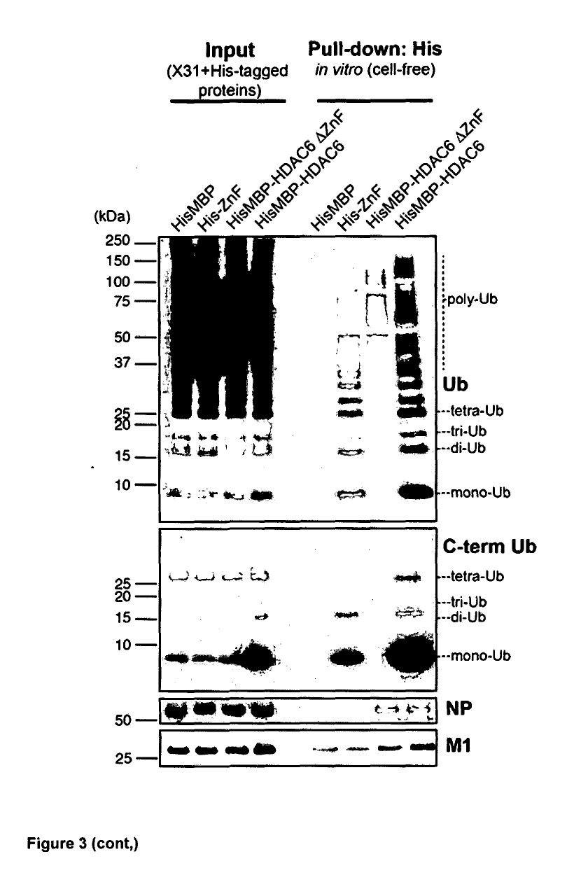

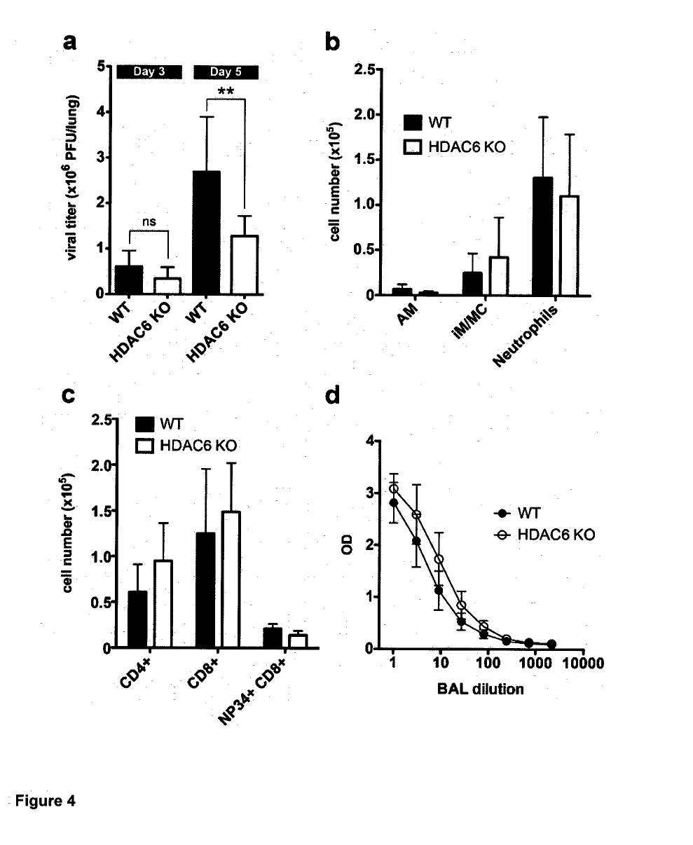

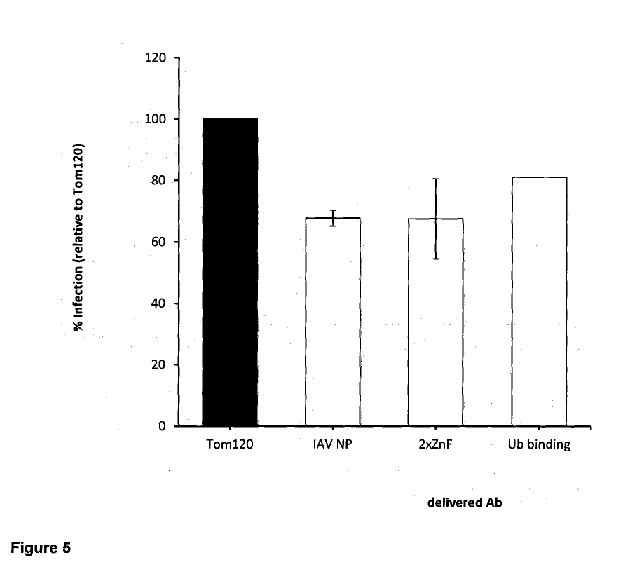

### DETAILED DESCRIPTION OF THE INVENTION

The present inventors found and show that in the absence of the deacetylase HDAC6, reduced virus titers are observed in vivo and in vitro. Moreover, the inventors found that whereas the deacetylase activity of HDAC6 is not important for the reduced titers, the ZnF-UBP domain of HDAC6, i.e. the ubiquitin-binding property of HDAC6, is required.

The present invention therefore provides a method for treating an influenza virus infection in a subject characterised in that a therapeutically effective amount of a modulator of the ubiquitin-binding property of HDAC6 is administered to said subject. In some embodiments, the modulator inhibits the ubiquitin-binding property of HDAC6. In some embodiments, the influenza virus is the influenza A virus. In some embodiments the modulator of HDAC6 decreases or silences the expression of HDAC6 and is, for example, a siRNA. In some embodiments, the modulator of HDAC6 is an antibody specifically binding to HDAC6, for instance a single-domain antibody.

The present invention also provides an antibody binding to HDAC6 and decreasing or blocking its ubiquitin-binding properties for use in treating an influenza virus infection. In some embodiments, this antibody is a single-domain antibody.

As used herein, the term “population” may be any group of at least two individuals. A population may include, e.g., but is not limited to, a reference population, a population group, a family population, a clinical population, and a same sex population.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified e.g. for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “reference standard population” means a population characterized by one or more biological characteristics, e.g., drug responsiveness, genotype, haplotype, phenotype, etc. As used herein, the term “subject” means that preferably the subject is a mammal, such as a human, but can also be an animal, including but not limited to, domestic animals (e.g., dogs, cats and the like), farm animals (e.g., cows, sheep, pigs, horses and the like) and laboratory animals (e.g., monkeys such as cynmologous monkeys, rats, mice, guinea pigs and the like).

As used herein, a “test sample” means a biological sample obtained from a subject of interest. For example, a test sample can be a biological fluid (e.g., serum), cell sample, or tissue, or isolated nucleic acid or polypeptide derived therefrom.

As used herein, the expression “body fluid” is a biological fluid selected from a group comprising blood, bile, blood plasma, serum, aqueous humor, amniotic fluid, cerebrospinal fluid, sebum, intestinal juice, semen, sputum, sweat and urine.

As used herein, the term “dysregulation” means a change that is larger or equal to 1.2 fold and statistically significant (p<0.05, Student's t-test) from the control. For example, a 1.5, 2, 2.5, 3, 3.5, 4, 4.5, or 5 fold change.

As used herein, the term “statistically significant” means a p value <0.05 as compared to the control using the Student's t-test.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies and technologies where quantitation of amplified species occurs subsequent to amplification, such as array technologies.

Array technologies involve the hybridisation of samples, representative of gene expression within the subject or control sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonuleotide arrays may be synthesised in situ (e.g by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech). Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a subject may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those listed above and in Table 2. Accordingly it will be recognised that such oligonucleotide arrays may be particularly preferred in embodiments of the methods of the invention where it is desired to compare expression of more than one gene of the invention.

Other suitable methodologies that may be used in the comparison of nucleic acid targets representative of gene expression include, but are not limited to, nucleic acid sequence based amplification (NASBA); or rolling circle DNA amplification (RCA).

The term “epitopes,” as used herein, refers to portions of a polypeptide having antigenic or immunogenic activity in an animal, in some embodiments, a mammal, for instance in a human. In an embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immuno specifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Fragments which function as epitopes may be produced by any conventional means. (See, e.g., Houghten, Proc. Natl. Acad. Sci. USA 82:5131-5135 (1985), further described in U.S. Pat. No. 4,631,211).

As one of skill in the art will appreciate, and as discussed above, polypeptides comprising an immunogenic or antigenic epitope can be fused to other polypeptide sequences. For example, polypeptides may be fused with the constant domain of immunoglobulins (IgA, IgE, IgG, IgM), or portions thereof (CHI, CH2, CH3, or any combination thereof and portions thereof), or albumin (including but not limited to recombinant albumin (see, e.g., U.S. Pat. No. 5,876,969, issued Mar. 2, 1999, EP Patent 0 413 622, and U.S. Pat. No. 5,766,883, issued Jun. 16, 1998)), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988).

Enhanced delivery of an antigen across the epithelial barrier to the immune system has been demonstrated for antigens (e.g., insulin) conjugated to an FcRn binding partner such as IgG or Fc fragments (see, e. g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Blochem., 270:3958-3964 (1995). Nucleic acids encoding the above epitopes can also be recombined with a gene of interest as an epitope tag (e.g., the hemagglutinin (“HA”) tag or flag tag) to aid in detection and punification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni²⁺ nitriloacetic acid-agarose column and histidine-tagged proteins can be selectively eluted with imidazole-containing buffers. Additional fusion proteins may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834, 252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998).

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), single-domain antibodies (sdAb, also called nanobodies), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgGI, IgG2, IgG3, IgG4, IgAI and IgA2) or subclass of immunoglobulin molecule. In addition, in the context of the present invention, the term “antibody” shall also encompass alternative molecules having the same function, e.g. ankyrin repeats, aptamers and/or CDRs grafted onto alternative peptidic or non-peptidic frames. In some embodiments the antibodies are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CHI, CH2, and CH3 domains. The antibodies of the invention may be from any animal origin including birds and mammals. In some embodiments, the antibodies are human, murine (e.g., mouse and rat), donkey, ship rabbit, goat, guinea pig, camel, shark, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al. The antibodies of the present invention may be monospecific, bispecific, trispecific or of greater multi specificity. Multispecific antibodies may be specific for different epitopes of a polypeptide or may be specific for both a polypeptide as well as for a heterologous epitope, such as a heterologous polypeptide or solid support material. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide which they recognize or specifically bind. The epitope(s) or polypeptide portion(s) may be specified as described herein, e.g., by N-terminal and C-terminal positions, by size in contiguous amino acid residues. Antibodies may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide are also included in the present invention.

Antibodies may also be described or specified in terms of their binding affinity to a polypeptide Antibodies may act as agonists or antagonists of the recognized polypeptides. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation, for instance an antibody against the ZnF-UBP domain of HDAC6, as well as antibodies that recognize the receptor-ligand complex. Likewise, encompassed by the invention are antibodies which bind the ligand, thereby preventing receptor activation, but do not prevent the ligand from binding the receptor. The antibodies may be specified as agonists, antagonists or inverse agonists for biological activities comprising the specific biological activities of the peptides disclosed herein. The above antibodies can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(I):14-20 (1996).

As discussed in more detail below, the antibodies may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396, 387. The antibodies as defined for the present invention include derivatives that are modified, i. e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen.

Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvurn. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art.

Antibody fragments which recognize specific epitopes may be generated by known techniques. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CHI domain of the heavy chain.

For example, the antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. As described in these references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax. et al., BioTechniques 12(6):864-869 (1992); and Sawai et al., AJRI 34:26-34 (1995); and Better et al., Science 240:1041-1043 (1988).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816397. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, and/or improve, antigen binding. These framework substitutions are identified by methods well known in the art, e.g., by modelling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988).) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592, 106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immurnol. 13:65-93 (1995). For a detailed discussion of this technology for producing human antibodies and human monoclonal antibodies and protocols for producing such antibodies, see, e. g., PCT publications WO 98/24893; WO 92/01047; WO 96/34096; WO 96/33735; European Patent No. 0 598 877; U.S. Pat. Nos. 5,413,923; 5,625,126; 5,633,425; 5,569, 825; 5, 661,016; 5,545,806; 5,814,318; 5,885,793; 5,916,771; and 5,939,598. In addition, companies such as Abgenix, Inc. (Freemont, Calif.) and Genpharm (San Jose, Calif.) can be engaged to provide human antibodies directed against a selected antigen using technology similar to that described above. Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)). Furthermore, antibodies can be utilized to generate anti-idiotype antibodies that “mimic” polypeptides using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization. and/or binding of a polypeptide to a ligand can be used to generate anti-idiotypes that “mimic” the polypeptide multimerization. and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide and/or to bind its ligands/receptors, and thereby block its biological activity. Polynucleotides encoding antibodies, comprising a nucleotide sequence encoding an antibody are also encompassed. These polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

The amino acid sequence of the heavy and/or light chain variable domains may be inspected to identify the sequences of the complementarity determining regions (CDRs) by methods that are well known in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and in some embodiments, human framework regions (see, e.g., Chothia et al., J. Mol. Biol. 278: 457-479 (1998) for a listing of human framework regions). In some embodiments, the polynucleotide generated by the combination of the framework regions and CDRs encodes an antibody that specifically binds a polypeptide. In some embodiments, as discussed supra, one or more amino acid substitutions may be made within the framework regions, and, in some embodiments, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polymicleotide are encompassed by the present description and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)). The present invention encompasses antibodies recombinantly fused or chemically conjugated (including both covalently and non-covalently conjugations) to a polypeptide (or portion thereof, in some embodiments, at least 10, 20, 30, 40, 50, 60, 70, 80, 90 or 100 amino acids of the polypeptide) to generate fusion proteins. The fusion does not necessarily need to be direct, but may occur through linker sequences. The antibodies may be specific for antigens other than polypeptides (or portion thereof, in some embodiments, at least 10, 20, 30, 40, 50, 60, 70, 80, 90 or 100 amino acids of the polypeptide). Further, an antibody or fragment thereof may be conjugated to a therapeutic moiety, for instance to increase their therapeutical activity. The conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, a-interferon, B-interferon, nerve growth factor, platelet derived growth factor, tissue plasminogen activator, an apoptotic agent, e.g., TNF-alpha, TNF-beta, AIM I (See, International Publication No. WO 97/33899), AIM 11 (See, International Publication No. WO 97/34911), Fas Ligand (Takahashi et aL, Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 interleukin-2 (“IL-2”), interleukin-6 (“IL-6”), granulocyte macrophage colony stimulating factor (“GM-CSF”), granulocyte colony stimulating factor (“G-CSF”), or other growth factors. Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

The present invention is also directed to antibody-based therapies which involve administering antibodies of the invention to an animal, in some embodiments, a mammal, for example a human, patient to treat influenza virus infections. Therapeutic compounds include, but are not limited to, antibodies (including fragments, analogs and derivatives thereof as described herein) and nucleic acids encoding antibodies of the invention (including fragments, analogs and derivatives thereof and anti-idiotypic antibodies as described herein). Antibodies of the invention may be provided in pharmaceutically acceptable compositions as known in the art or as described herein.

In some embodiments, said inhibitory compound is a small molecule, an antibody or a siRNA. In an embodiment, the compound is substantially purified (e.g., substantially free from substances that limit its effect or produce undesired side-effects). The subject is in some embodiments, an animal, including but not limited to animals such as cows, pigs, horses, chickens, cats, dogs, etc., and is in some embodiments, a mammal, for example human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound, e.g., encapsulation in liposomes, microparticles, microcapsules, recombinant cells capable of expressing the compound, receptor-mediated endocytosis (see, e. g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.) In yet another embodiment, the compound or composition can be delivered in a controlled release system. In one embodiment, a pump may be used (see Langer, supra; Sefton, CRC Crit. Ref, Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g. Goodson, in Medical Applications of Controlled Release, supra, vol. 2, pp. 115-13 8 (1984)). Other controlled release systems are discussed in the review by Langer (Science 249:1527-1533 (1990)).

The present invention also provides pharmaceutical compositions for use in the treatment of influenza. Such compositions comprise a therapeutically effective amount of an inhibitory compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, tale, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, in some embodiments, in purified form, together with a suitable amount of carrier so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lidocaine to ease pain at the site of the injection.

Generally, the ingredients are supplied either separately or mixed together in unit dosage form, for example, as a dry lyophilized powder or water free concentrate in a hermetically scaled container such as an ampoule or sachette indicating the quantity of active agent.

Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms.

Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the compound which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patients circumstances.

Effective doses may be extrapolated from dose-response curves derived from in vitro or animal model test systems. For antibodies, the dosage administered to a patient is typically 0.1 mg/kg to 100 mg/kg of the patients body weight. In some embodiments, the dosage administered to a patient is between 0.1 mg/kg and 20 mg/kg of the patients body weight, for example 1 mg/kg to 10 mg/kg of the patients body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

Also encompassed is a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The antibodies as encompassed herein may also be chemically modified derivatives which may provide additional advantages such as increased solubility, stability and circulating time of the polypeptide, or decreased immunogenicity (see U.S. Pat. No. 4,179,337). The chemical moieties for derivatisation may be selected from water soluble polymers such as polyethylene glycol, ethylene glycol/propylene glycol copolymers, carboxymethyl cellulose, dextran, polyvinyl alcohol and the like.

The antibodies may be modified at random positions within the molecule, or at predetermined positions within the molecule and may include one, two, three or more attached chemical moieties. The polymer may be of any molecular weight, and may be branched or unbranched. For polyethylene glycol, the preferred molecular weight is between about 1 kDa and about 100000 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,600, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999). The polyethylene glycol molecules (or other chemical moieties) should be attached to the protein with consideration of effects on functional or antigenic domains of the protein. There are a number of attachment methods available to those skilled in the art, e.g., EP 0 401 384 (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group. As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein. As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

“siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein. In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity. Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYL1P. The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among: a) a 3′ cap; b) a 5′ cap, c) a modified internucleoside linkage; or d) a modified sugar or base moiety.

Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates.

End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference.

The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi.

It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators).

The gene-silencing molecule may be either synthesised de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell.

The molecule may be produced by a vector harbouring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesise the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favour targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like).

Alternatively a “naked” siRNA or antisense molecule may be inserted into a subject's cells by a suitable means e.g. direct endocytotic uptake.

The gene silencing molecule may also be transferred to the cells of a subject to be treated by either transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product). Inhibitors according to any embodiment of the present invention may be used in a monotherapy (e.g. use of siRNAs alone). However it will be appreciated that the inhibitors may be used as an adjunct, or in combination with other therapies.

The antagonist of HDAC6 may be contained within compositions having a number of different forms depending, in particular on the manner in which the composition is to be used. Thus, for example, the composition may be in the form of a capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and in some embodiments, enables delivery of the inhibitor to the target site.

The antagonist of HDAC6 may be used in a number of ways.

For instance, systemic administration may be required in which case the compound may be contained within a composition that may, for example, be administered by injection into the blood stream. Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted in the body of the subject, and the molecule may be released over weeks or months. Such devices may be particularly advantageous when long term treatment with an antagonist of HDAC6 is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the subject being treated.

Optimal dosages to be administered may be determined by those skilled in the art, and will vary with the particular inhibitor in use, the strength of the preparation, and the mode of administration. Additional factors depending on the particular subject being treated will result in a need to adjust dosages, including subject age, weight, gender, diet, and time of administration.

When the inhibitor is a nucleic acid conventional molecular biology techniques (vector transfer, liposome transfer, ballistic bombardment etc) may be used to deliver the inhibitor to the target tissue. Known procedures, such as those conventionally employed by the pharmaceutical industry (e.g. in vivo experimentation, clinical trials, etc.), may be used to establish specific formulations for use according to the invention and precise therapeutic regimes (such as daily doses of the gene silencing molecule and the frequency of administration).

Generally, a daily dose of between 0.01 μg/kg of body weight and 0.5 g/kg of body weight of an antagonist of HDAC6 may be used for the treatment of influenza infections in the subject, depending upon which specific inhibitor is used. When the inhibitor is an siRNA molecule, the daily dose may be between 1 pg/kg of body weight and 100 mg/kg of body weight, in some embodiments, between approximately 10 pg/kg and 10 mg/kg, or between about 50 pg/kg and 1 mg/kg.

When the inhibitor (e.g. siNA) is delivered to a cell, daily doses may be given as a single administration (e.g. a single daily injection).

Various assays are known in the art to test dsRNA for its ability to mediate RNAi (see for instance Elbashir et al., Methods 26 (2002), 199-213). The effect of the dsRNA according to the present invention on gene expression will typically result in expression of the target gene being inhibited by at least 10%, 33%, 50%, 90%, 95% or 99% when compared to a cell not treated with the RNA molecules according to the present invention.

Similarly, various assays are well-known in the art to test antibodies for their ability to inhibit the biological activity of their specific targets. The effect of the use of an antibody according to the present invention will typically result in biological activity of their specific target being inhibited by at least 10%, 33%, 50%, 90%, 95% or 99% when compared to a control not treated with the antibody.

The term inhibiting or antagonist (compound/agent) as used herein refers to a molecule which, when binding or interacting with a protein, or with functional fragments thereof, decreases the intensity or extends the duration of the biological activity of said protein. This definition further includes those compounds that decrease the expression of the gene coding for said protein. An inhibiting agent may be made up of a peptide, a protein, a nucleic acid, carbohydrates, an antibody, a chemical compound or any other type of molecule decreasing the effect and/or the duration of the activity of the target protein.

HDAC6, also known as histone deacetylase 6, EC 3.5.1.983, HD6, JM211, FLJ16239, OTTHUMP00000032398, KIAA0901, or OTTHUMP00000197663, plays a central role in microtubule-dependent cell motility via deacetylation of tubulin. In addition to its protein deacetylase activity, HDAC6 binds with high affinity ubiquitin or ubiquitinated proteins and plays a key role in the degradation of misfolded proteins, i.e. when misfolded proteins are too abundant to be degraded by the chaperone refolding system and the ubiquitin-proteasome, HDAC6 mediates the transport of misfolded proteins to the aggresome, a cytoplasmic juxtanuclear structure and also promotes the formation of stress granules. HDAC6 belongs to class IIb of the histone deacetylase/acuc/apha family. It contains an internal duplication of two catalytic domains which appear to function independently of each other. This protein possesses histone deacetylase activity and can repress transcription if present in the nucleus. Additional known substrates of HDAC6 are the chaperone Hsp90 or the actin-binding protein cortactin. In some experiments HDAC6 has been shown to deacetylate the N-terminal tails of histones. Histone deacetylation gives a tag for epigenetic repression and plays an important role in transcriptional regulation, cell cycle progression and developmental events.

The HDAC6 gene is expressed relatively ubiquitously and is not known to be induced in response to stimuli. It has been shown that acetylation of HDAC6 by p300 attenuates its deacetylase activity (Han Y et al., 2009). Also, Aurora kinase A (AurA) colocalizes with HDAC6 at the basal body of cilia and phosphorylates it, thereby enhancing its tubulin deacetylase activity (Pugacheva et al., 2007). Furthermore, it was also shown that protein kinase CKII phosphorylates HDAC6 on Serine 458, increasing its deacetylase activity and promoting formation and clearance of aggresomes (Watabe and Nakaki, 2012).

As used herein, the “enzymatic activity of HDAC6” refers to the enzymatic (deacetylase) activity of HDAC6, whereas the capacity of HDAC6 to bind ubiquitinated proteins is referred to as “ubiquitin-binding activity of HDAC6” or “ubiquitin-binding property of HDAC6”.

In the context of the present invention, i.e. for the treatment of influenza infections, the terms “antagonist of HDAC6” or “inhibitors of HDAC6” refers to agents/molecules which specifically block or strongly reduce the ubiquitin-binding activity of HDAC6.

Influenza, commonly known as “the flu”, is an infectious disease of birds and mammals caused by RNA viruses of the family Orthomyxoviridae, the influenza viruses.

The Orthomyxoviruses (orthos, Greek for “straight”; myxa, Greek for “mucus”) are a family of RNA viruses that includes six genera: Influenza virus A, Influenza virus B, Influenza virus C, Isavirus, Thogotovirus and a recently discovered, still undescribed genus. The first three genera contain viruses that cause influenza in vertebrates, including birds (see also avian influenza), humans, and other mammals. Isaviruses infect salmon; thogotoviruses infect vertebrates and invertebrates, such as mosquitoes and sea lice.

The Influenza viruses A, B and C, which are identified by antigenic differences in their nucleoprotein and matrix protein, infect vertebrates as follows: Influenza virus A infects humans, other mammals, and birds, and causes all flu pandemics; Influenza virus B infects humans and seals; Influenza virus C infects humans and pigs.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Examples

### Cells and Viruses

A549 cells were obtained from the American Type Culture Collection, and mouse embryonic fibroblasts (MEFs) were isolated from embryonic day 13.5 from male embryos of the wild type (WT) and HDAC6 knockout (KO) mice. The WT MEFs and rescue cell-lines expressing HDAC6 WT or mutants were established following standard protocols, as described (Zhang, Gilquin et al. 2006). To generate the HDAC6 (Db^{m}) rescue cell-line, the HDAC6 KO MEFs were infected with pMSCV-IRES-GFP plasmid containing mouse HDAC6 gene without the dynein-binding domain, i.e. the linker region between the two catalytic domains (de1437-481). Deletion construct was made with QuickChange Mutagenesis Kit (Agilent Technologies). All cells were maintained in Dulbecco's modified Eagle's medium (D-MEM) (Invitrogen), supplemented with 10% fetal calf serum, 1% non-essential amino acids, and antibiotics under 5% CO₂ at 37° C. Influenza A X31 virus strain (an H3N2 reassorted strain derived from the A/Puerto Rico/8/34 (PR8) and A/Hong Kong/1/68 strains) was purchased from Virapur (CA, USA) in purified form. The titer of the virus stock, determined in MDCK cells, was 2.4×10⁵ TCID₅₀ infectious units/μl.

### Antibodies, Plasmids, and Reagents

Anti-HA monoclonal, ployclonal (Pinda), A1, M1 (HB64), NP (HB65) antibodies were used, as described (Banerjee, Yamauchi et al. 2013). The other antibodies used in this study were: anti-HDAC6 (rabbit polyclonal antibody, produced in the lab of Dr. Patrick Matthias, FMI Basel), anti-Ubiquitin (Santa Cruz Biotechnology), anti-C-terminal ubiquitin (Millipore), PCNA (Santa Cruz Biotechnology), anti-α-Tubulin (Sigma), anti-acetylated-α-Tubulin (Sigma) anti-LAMP1 (Abcam), anti-dynactin 2 (Paschal, Holzbaur et al. 1993), anti-Myosin IIa, b (Cell Signaling). Hoechst 33258, DAPI, Wheat Germ Agglutinin (WGA)-Alexa Fluor 647, Phalloidin-Alexa Fluor 647, R18, and SP-DiOC18(3) were purchased from Invitrogen. DRAQS was purchased from Biostatus Limited. DMSO, Bafilomycin A1 (BafA1), cycloheximide, ammonium chloride, ML-9, blebbistatin (Blebb), cytochalasin D (CytoD), nocodazole, MG132, importazole (Ipz) fibronectin, PMSF proteinase inhibitor and formaldehyde solution (36%) were purchased from Sigma-Aldrich. Proteinase K was purchased from Promega. cOmplete protease inhibitor cocktail tablets were purchased from Roche. siRNA transfections siRNAs (AllStars, HDAC6_oligo 1 (CACCGTCAACGTGGCATGGAA; SEQ ID NO:1), HDAC6_oligo 2 (CACTTCGAAGCGAAATATTAA; SEQ ID NO:2), ATP6V1B2_3 (CACGGTTAATGAAGTCTGCTA; SEQ ID NO:3), and DCTN2_3 (AACGAGATTGCCAAGCATA; SEQ ID NO:4) were purchased from Qiagen. MYH9 (GAGTCTGAGCGTGCTTCCAGGAATA; SEQ ID NO:5) and MYL10 (GGAGACCATTCTCCACGCCTTCAAA; SEQ ID NO:6) (Hao, Nanduri et al. 2013) were purchased from Microsynth AG. Reverse transfection on A549 cells was carried out with Lipofectamine RNAiMax (Invitrogen) and D-MEM. A final concentration of 10 nM siRNA was used for transfection. After transfection, the cells were kept in a 5% CO₂ incubator at 37° C. for 3 days, following which experiments were performed.

### IAV Entry and Infection Assays

All virus assays were performed in infection medium, consisting of D-MEM with 50 mM HEPES buffer and 0.2% BSA, pH 6.8. The assays for endocytosis, HA acidification, fusion, uncoating, nuclear import, and infection were carried out as per the protocols described (Banerjee, Yamauchi et al. 2013). The detection time-points for each of the entry assays were: 30 min for endocytosis, 1 h for HA acidification, 1.5 h for fusion, 2.5 h for uncoating, 4 h for nuclear import, and 10 h for infection (NP synthesis). The assays were performed either in 24-well plates for high-resolution confocal imaging (using 100× and 63× objectives) and FACS analysis, whereas for high-throughput analysis, 96-well optical bottom plates (Greiner) were used. Before the entry assays with MEFs, the glass coverslips in 24-well plates and the surface of the wells of the 96-well plates were coated with fibronectin (50 μg/ml in PBS) for 30 min. Coverslips were mounted on slides by using Immomount (Thermo Fisher Scientific). High-resolution images were acquired on an inverted confocal microscope (Zeiss LSM 510 Meta), and automated image acquisition of 96-well plates was performed with a 20× objective using Molecular Devices ImageXpress Micro imaging system. 9 images (3×3) were acquired from each well for each channel. Alternatively, NP-positive cells were analyzed by FACS.

### Acid-Induced Fusion Assay at the Plasma Membrane

Labeling of the virus with fluorescent dyes (R18/SP-DiOC(18)) was done as described (Sakai, Ohuchi et al. 2006). In brief, 50 μl of virus stock was diluted in 750 μl PBS, to which a premix of R18 and SP-DiOC18(3) was added with vigorous mixing, at a final concentration of 0.4 μM and 0.2 μM, respectively. The mixture was rocked for 1 h at room temperature in the dark, and filtered through a syringe filter with a 0.22 μm pore size (Millipore) to remove unbound dye and aggregates. Cells were trypsinised and 50,000 cells were taken in each eppendorf tube. 50 μl of the labelled virus was mixed with 50 μl infection medium and bound on ice for 30 min. Cells were washed with cold infection medium to remove unbound virus particles. After removal of the supernatant, 300 μl of pre-warmed pH 5.0/pH 7.4 media were added to the cells and incubated at 37° C. for 1 min. Cells were fixed immediately by adding 300 μl of 8% formaldehyde. After washing, the cells were resuspended in 250 μl of FACS buffer, and analyzed by FACS.

### Acid-Bypass Uncoating and Infection Assays

To detect M1 uncoating and NP expression by inducing viral fusion at the plasma membrane, acid-bypass uncoating and infection assays were performed. Virus stock (10 μL for uncoating assay and 0.25 μL for infection assay/well of 96-well plate) was prebound to cells at 4° C. for 1 h, after which the inoculum was removed. Cells were washed once with ice-cold infection medium on ice and replaced by either prewarmed pH 7.4 medium or with low pH containing infection medium, buffered to pH 5.0 with 50 mM citrate buffer. Cells were incubated at 37° C. in water bath for 2 min, washed 2 times with cold infection medium, and the infection medium was replaced with STOP medium (DMEM with 50 mM HEPES, pH adjusted to 7.4 and supplemented with 20 mM NH₄Cl). Cells were further incubated at 37° C. for 2 min for uncoating assay or 8 h for infection assay with 5% CO₂, after which, they were fixed, and processed for microscopy. Alternatively, M1-Positive Cells were Analyzed by FACS.

### Proteinase K Sensitivity Assay

Purified X31 was lysed with lysis buffer with or without 0.1% triton X-100 on ice for 1 h. Different concentrations of proteinase K (1, 2, 5 mg/ml) were added to each sample and incubated at room temperature for 15 min. The digestion reaction by proteinase K was stopped by adding PMSF (2 mM final concentration) and Laemmli sample buffer (5×). Western blotting was done to detect ubiquitin, NP and M1.

### HDAC6 Pull-Down Assay

His-tagged constructs (HisMBP, His-Zn F, HisMBP-HDAC6-ΔZnF and HisMBP-HDAC6), were expressed in Sf9 cell-line from Spodoptera frugiperda and purified by Ni-NTA agarose affinity chromatography matrix (Qiagen) and gel filtration. His-tagged ZnF-UBP domain of HDAC6 was expressed in E. coli, and purified with the same method. Purified X31 was lysed in lysis buffer (10 mM PIPES pH 6.8, 300 mM sucrose, 100 mM NaCl, 3 mM EGTA, 0.1% (v/v) triton X-100, 1× cOmplete protease inhibitor), and mixed with purified His-tagged proteins in a buffer containing 20 mM Tris-HCl pH 7.5, 100 mM NaCl and 0.1% Triton X-100. The mixture was incubated at 4° C. overnight after which, NiNTA agarose beads and imidazole (at 20 mM final concentration) were added to the mix, and further incubated at 4° C. for 30 min. The beads were then spun down by centrifugation at 1500 g for 2 min, and the precipitated beads were washed twice with wash buffer-1 (20 mM Tris-HCl pH7.5, 100 mM NaCl, 0.1% Triton X-100, 20 mM imidazole and 1% BSA) and twice with wash buffer-2 (20 mM Tris-HCl pH7.5, 100 mM NaCl). Laemmli sample buffer (5×) was added to the beads, and after heating at 95° C. for 2 min, the samples were loaded onto a 4-12% NuPAGE precast polyacrylamide gel (Invitrogen), and run for 36 min at 200V. Western blotting was done to detect His-tag, HDAC6, ubiquitin, unanchored ubiquitins with exposed C-terminal ends, NP, and M1.

### Immunoprecipitation Assay

Five hundred μl of the virus stock, diluted in 14.5 ml infection medium with 1 mM cycloheximide, was added to a 15 cm petri dish with ˜80% confluent cells. The cells were then incubated at 37° C. for 2.5 h, washed with ice-cold PBS 3 times, and lysed with 250 μl of IP-buffer (20 mM Tris pH 7.5, 150 mM NaCl, 5 mM MgCl₂, 0.5% NP-40, 10% glycerol, 4 mM NaF, 2 mM β-glycerophosphate, 200 μM sodium vanadate, 1 mM DTT, and cOmplete, EDTA-free proteinase inhibitor). Cell lysis was performed on ice by passing them 3 times through a 27 G needle. After incubation on ice for 30 min, the cells were centrifuged at 16,000 g for 30 min to remove insoluble material. The protein concentration in the lysate was measured using the Bradford assay. Ten % of the sample was taken and boiled for 10 min at 95° C. This sample served as input. Four μl of rabbit anti-HDAC6 antibody was added and incubated at 4° C. on a rotating wheel overnight. Protein-G sepharose beads (GE Healthcare) was washed 3 times with the IP-buffer and 20 μl of a 50% protein-G bead slurry was added to the lysate and incubated for an additional 1 h at 4° C. on a rotating wheel. The beads were then washed 5 times with 700 μl IP-buffer. The samples were then boiled at 95° C. in 50 μl sample buffer for 10 min.

### IAV Infection in HDAC6 KO Mice

C57BL/6 WT mice were purchased from Charles River (Germany). HDAC6−/− mice were generated in the lab of Patrick Matthias, FMI. All animals were housed in individually ventilated cages under specific pathogen free conditions at BioSupport AG (Zurich, Switzerland) and used for experiments when between 8 and 12 weeks of age. All animal experiments were performed according to institutional guidelines and Swiss federal regulations. All animal experiments had been approved by Swiss federal and local animal ethics committees. To carry out infection, the WT and the HDAC6 KO mice were anaesthetized and intra-tracheally inoculated with 50 μL of PR8 strain of IAV (A/Puerto Rico/34, H1N1) in endotoxin-free PBS of the indicated doses. To determine influenza viral titers in the lungs, samples were collected on various days after infection, homogenized and serially diluted with MDCK cells as described (Bachmann, Ecabert et al. 1999) using anti-NP antibody (HB65). Virus-specific antibodies were detected by ELISA using recombinant PR8 HA (a kind gift of M. Bachmann, Cytos).

### FACS Analysis

Multiparameter analysis was performed on a FACSCanto II (Becton Dickinson Immunocytometry Systems) and analyzed with FlowJo software (Tree Star). Monoclonal antibodies specific to mouse CD11c (N418), CD11b (M1/70), Ly-6C (HK1.4), Siglec-F (E50-2440, BD Biosciences), CD115 (AFS98, eBioscience), CD45 (30-F11), CD4 (GK1.5), CD8alpha (53-6.7), Gr-1 (RB6-8C5, eBioscience) were purchased from Biolegend unless otherwise stated. Dead cells were stained using eFluor780 (eBioscience). PE-conjugated peptide-MHC class I tetramers (H-2Db/NP34) with the peptide NP34 (NP366-374; ASNENMETM) from the nucleoprotein of influenza virus A/PR/8/34 were generated as described (Altman, Moss et al. 1996). For intracellular cytokine staining, 1.5×10⁵ bone marrow-derived dendritic cells (BMDC) were incubated overnight with 1×10⁶ pfu UV-inactivated PR8 virus in 96-well plates and pulsed with 1 μg/mL NP34 peptide for 2 hours before BAL or from individual mice were added. Restimulation was performed for 4-5 h in the presence of 2 μM monensin (Sigma-Aldrich). After surface staining and formalin-fixation, intracellular cytokine staining was done in the presence of 0.5% saponin using anti-mouse TNF-alpha (MP6-XT22) and IFN-gamma (XMG1.1). Prior to all stainings, FcgammaIII/II receptors were blocked by incubation with homemade anti-CD16/32 (2.4G2). For acidification and uncoating assays, fixed cells were stained with monoclonal A1 antibody (1:1000 dilution) and HB64 antibody (1:2000 dilution), respectively. The cells were then stained with anti-mouse secondary antibodies conjugated with fluorophore (1:2000 dilution). Statistical analysis Data are represented as mean±SD. For all analyses, multiple independent experiments (N≧3) were carried out. Student's t-tests were performed for all analyses using Prism 6 (GraphPAD Software Inc.) software. Statistical significance was determined by two-tailed P values: ns P>0.05, *P<0.05, **P<0.01, ***P<0.001.

### HDAC6 is Required for Capsid Uncoating

The present inventors found that siRNA-mediated depletion of histone deacetylase 6 (HDAC6) in A549 cells (a human bronchial epithelial cell-line) reduced infectivity of the X31 strain of IAV (an H3N2 reassorted strain derived from the A/Puerto Rico/8/34 (PR8) and A/Hong Kong/1/68 strains) to about 50%, and that infection in HDAC6 knock-out (KO) mouse embryo fibroblasts (MEFs) was reduced to 30% as compared to wild-type (WT) MEFs. Infection was measured as the fraction of cells expressing newly synthesized NP, detected by indirect immunofluorescence (IIF)-based assay.

HDAC6 is a class II HDAC predominantly found in the cytoplasm (Hubbert, Guardiola et al. 2002). It has two functional deacetylase domains and a C-terminal zinc-finger-containing domain (ZnF-UBP) that binds mono- and polyubiquitin (mono- and poly-Ub) with high affinity (True and Matthias 2012). The linker region between the two deacetylase domains mediates dynein motor binding for the transport of misfolded, ubiquitinated proteins to aggresomes (Kawaguchi, Kovacs et al. 2003). HDAC6 is known to regulate cell motility, adhesion, chemotaxis, and protein transport by deacetylating microtubules (MTs), Hsp90, and cortactin (Yang and Seto 2008; True and Matthias 2012).

The inventors analyzed the IAV infection process in WT and HDAC6 KO MEFs using an array of quantitative assays to monitor consecutive steps in entry (Banerjee, Yamauchi et al. 2013). No significant difference was observed between the WT and HDAC6 KO MEFs in the endocytic uptake of IAV, acid-induced conversion of the HA in late endosomes (LEs), and fusion of the viral and endosomal membranes. This demonstrated that the initial steps including penetration into the cytosol were likely to be unaffected by the loss of HDAC6.

However, nucleocapsid uncoating and the subsequent step, nuclear import of vRNPs, were reduced in HDAC6 KO MEFs to 22% and 17%, respectively. IIF showed that instead of being dispersed throughout the cytoplasm, M1 was present in cytoplasmic spots with a distribution similar to LEs. The spots were positive for LAMP1, a LE/lysosome marker. HDAC6 depletion by siRNA in A549 cells showed similar effects; IAV uncoating was reduced to 21% of the cells compared to the control, while HA acidification and fusion were unaffected. The results suggested that after fusion at LEs, HDAC6 plays a role in the release of viral capsids from the cytosolic surface of endosomes, the dissociation of M1 from vRNPs, and the dispersion of capsid components in the cytosol.

To confirm that the effect was in fact post-fusion, the present inventors tested whether the HDAC6 requirement could be bypassed by acid-induced IAV fusion and capsid penetration through the plasma membrane (PM). Fusion of the virus with the PM allows infectious delivery of viral capsids into the cytosol without endocytosis (Matlin, Reggio et al. 1981; White, Matlin et al. 1981; Yamauchi, Boukari et al. 2011). The inventors allowed the virus particles to bind on the cell surface at pH 6.8 in the cold, and then briefly dropped the medium pH to 5.0 at 37° C. After induction of fusion, ammonium chloride was added to the medium to prevent virus entry through the endosome route.

Following acid-bypass, no difference in viral fusion activity was observed between the WT and HDAC6 KO MEFs, but M1 uncoating was reduced to 32% in the HDAC6 KO MEFs and to 47% in HDAC6-depleted A549 cells. This confirmed that the block involved post-fusion events. IIF staining of M1 indicated that uncoating was blocked. In HDAC6 KO MEFs following acid-bypass, M1 staining remained punctate instead of the diffuse cytoplasmic staining observed in WT cells.

### The ZnF-UBP Domain of HDAC6 is Critical

Since HDAC6 has multiple functional domains, the inventors assessed which of them was required. For this, three rescue MEF cell-lines established in the HDAC6 KO background were used Kwon et al., 2007; Li et al. 2003). The cells had been rescued either with WT HDAC6 (WT^{r}), with HDAC6 that is catalytically inactive (HD^{m}), or defective for Ub-binding (ZnF^{m}) (Kwon et al., 2007).

When IAV was allowed to enter the HDAC6 (WT^{r}) and the HDAC6 (HD^{m}) cells, the inventors observed normal uncoating. However, uncoating, nuclear import, and infection were dramatically reduced in the HDAC6 (ZnF^{m}) cells. None of the three cell-lines showed a defect in HA acidification. In fact, HA acidification in the HDAC6 (ZnF^{m}) cells was elevated by 32% compared to WT MEFs. Following acid-bypass via the PM, uncoating was normal in cells rescued with HDAC6 (WT^{r}) and HDAC6 (HD^{m}), whereas it only reached a level of 40% in the HDAC6 (ZnF^{m}) cells. Similar results were obtained for infection. Taken together, the results revealed that the deacetylase activity of HDAC6 was not important for efficient disassembly of IAV capsids. However, the ZnF-UBP domain was required.

### HDAC6 Binds Free Ub Present Inside IAV

Mass spectrometry analysis of purified IAV (H1N1/WSN/33 strain) indicated the presence of Ub within the virion (Shaw, Stone et al. 2008). By western blotting we observed that purified X31 virions contained mono- and poly-Ub, some of which were unanchored di-, tri-, and tetra-Ub. To test whether the Ub molecules were present within the virion, the inventors subjected the purified X31 to proteinase K digestion with or without triton X-100. Only in the presence of triton X-100 the poly-Ub chains were digested. This indicated that, similar to M1 and NP, the Ub molecules were packaged inside the viral membrane.

To test whether HDAC6 interacted with the free Ub present within the virion and/or with viral proteins, the present inventors performed an in vitro pull-down assay. The full-length HDAC6, the HDAC6 ZnF-UBP domain, or HDAC6 lacking the ZnF-UBP domain (HDAC6 ΔZnF) were Histidine (His)-tagged and purified. The purified proteins were incubated with lysed X31, followed by pull-down using a Ni-NTA agarose matrix. The HDAC6 ZnF-UBP domain is known to bind exclusively to unanchored C-terminal diglycine motif of Ub (Ouyang, Ali et al. 2012). It binds Ub at its C-terminal Gly-Gly residues, unlike most other Ub-binding domains that recognize the Ub hydrophobic core (Ouyang, Ali et al. 2012). The HDAC6 ZnF-UBP domain pulled-down Ub molecules present inside the virion, which were verified as C-terminal free Ub using a specific antibody. Viral NP and M1 were also pulled-down by HDAC6 and their interaction with HDAC6 appeared to be independent of the ZnF-UBP domain. To test whether HDAC6 interacts with NP or M1 in cell culture, the inventors allowed IAV to enter WT and HDAC6 KO MEFs for 2.5 hours, and prepared lysates that we subjected to IP using a polyclonal anti-HDAC6 antibody. They found that NP and M1 co-immunoprecipitated with HDAC6.

When they examined the M1 staining pattern after IAV entry in the HDAC6 (ZnF^{m}) cells, M1 was detected in spots positive for LAMP1 with a distribution similar to that seen in HDAC6 KO cells. This suggested that the Ub-binding activity of HDAC6 promoted IAV capsid uncoating and their dissociation from LEs.

### Dynein and Myosin II Promote Uncoating

HDAC6 is known to interact with the dynein motor via p15^{glued}, a subunit of the dynactin complex. Using HDAC6 as an adaptor, dynein mediates the retrograde transport of misfolded protein aggregates along MTs to aggresomes (Kawaguchi, Kovacs et al. 2003). The inventors addressed whether dynein is required for IAV uncoating. Both siRNA-mediated depletion of dynactin 2, the p50 subunit of the dynactin complex, and pharmacological inhibition of dynein activity by ciliobrevin D (CilioD) (Firestone, Weinger et al. 2012), reduced uncoating following PM bypass to 51% and 62%, respectively. The inventors newly generated a HDAC6 rescue cell-line (Db^{m}), in which the expressed HDAC6 lacked the dynactin-binding domain i.e. the linker region between the two catalytic domains (Kawaguchi, Kovacs et al. 2003). Using PM bypass, they found that uncoating in the HDAC6 (Db^{m}) cells was reduced to 58%% of WT MEFs. When the virus was allowed to fuse at LEs, uncoating was reduced to 67% in HDAC6 (Db^{m}) cells compared to WT MEFs, while HA-acidification was unaffected. These results indicated that HDAC6 binding to dynein/dynactin promotes IAV uncoating. The dynein-HDAC6 interaction was confirmed by co-immunoprecipitation. IIF in the HDAC6 (Db^{m}) cells showed a similar pattern to the HDAC6 KO and (ZnF^{m}) cell-lines where M1 was detected within the LAMP1-positive vacuoles.

A recent study showed that HDAC6 binds to unanchored free Ub chains that are produced by the deubiquitination of ubiquitinated proteins in aggresomes. Binding to free Ub activates HDAC6 and stimulates the actomyosin system, after which the actin-associated motor protein type II non-muscle myosin 10 (IIB), but not myosin 9 (IIA), promotes deaggregation and clearance of the aggresome (Hao, Nanduri et al. 2013). Since X31 contained free unanchored Ub that bound to HDAC6 ZnF-UBP, the present inventors tested the role of the actomyosin system in uncoating. Using RNAi in A549 cells, they depleted myosin 9 and 10, which was confirmed by western blotting. After acid-bypass at the PM, virus uncoating in myosin 10-depleted cells decreased to 42% of the control whereas there was no effect in myosin 9-depleted cells. Pharmacological inhibition of type II myosin activity by the drugs ML-9 and blebbistatin (Blebb) blocked uncoating by more than half. When actin filaments were depolymerized with cytochalasin D (CytoD) uncoating was blocked by 83% following PM bypass. When MTs were depolymerized by nocodazole (Noc), uncoating decreased by 35%. When both types of cytoskeleton were depolymerized, uncoating dropped to 6%. In IAV-infected cells co-immunoprecipitation experiments showed that HDAC6 interacted with both myosin 10 and actin. Next the inventors examined whether MTs, dynein, actin, and myosin II facilitate uncoating when IAV was allowed to enter cells by endocytosis and to fuse at the LEs. After normal endocytic uptake of virus, endosome acidification was prevented with ammonium chloride (30 min to 90 min post-uptake), allowing the particles to accumulate within LEs. Drugs were added at 60 min post-uptake for 30 min, followed by ammonium chloride washout to allow viral fusion. To allow uncoating, the cells were incubated in the presence of drug for another 60 min. The results showed that drugs targeting MTs, dynein, actin, and myosin II all blocked uncoating. Inhibition of proteasome activity or importin-β-mediated nuclear import using MG132 and importazole (Ipz), respectively, had no significant effect (Soderholm, Bird et al. 2011). These results indicate that dynein and myosin II activity, intact MTs and actin cytoskeleton are essential for IAV uncoating, with HDAC6 linking the IAV capsid to these cellular components.

HDAC6 is Degraded in IAV Producer Cells Finally, the present inventors followed up an observation that HDAC6 is degraded in response to IAV infection (Husain and Harrod 2009). They could confirm the loss of HDAC6 roughly coinciding with the synthesis of new NP. The rational for the elimination of HDAC6 was probably the need for IAV to prevent interference from this uncoating factor during virus assembly and budding.

### HDAC6 Promotes IAV Infection In Vivo

Having identified HDAC6 as an uncoating factor for IAV in tissue culture cells, the present inventors determined whether it played a role during IAV infection in mice. WT and HDAC6 KO mice (Zhang et al., 2008) were infected intra-tracheally with the PR8 strain of IAV (A/Puerto Rico/34, H1N1), and viral titers in the lungs measured 3 and 5 days post-infection. In the HDAC6 KO mice, the lung viral titers were significantly reduced at day 5 compared to control mice. Similar results were obtained when the mice were infected with the X31 strain of IAV, the strain used in our in vitro experiments.

HDACs have been implicated in inflammation and immune responses in mice. HDAC6, in particular, has been shown to promote IFN-β production and immune synapse formation (Serrador, Cabrero et al. 2004; Nusinzon and Horvath 2006; Shakespear, Halili et al. 2011). Therefore, the inventors examined whether the observed reduction in the lung viral load was a consequence of an exaggerated immune response due to IAV infection. To assess the efficiency of the innate immune response, they measured the number of infiltrating inflammatory monocytes, neutrophils, and resident alveolar macrophages in the lungs. No significant difference in the innate immune response was observed between the WT and HDAC6 KO mice. Furthermore, the antiviral T cell response was comparable since the numbers of total CD4+ and CD8+, and the virus-specific NP34+ and CD8+ T cells were similar. There was no difference in the IFN-γ production of virus-specific CD8+ T cells and in the different isotypes of the IAV-specific antibody response between the WT and HDAC6 KO mice. Together, these data show that the reduced virus titers observed in the HDAC6 KO mice were not due to an altered antiviral immune response, but rather an impaired ability of the virus to infect or propagate in vivo.
Altman, J. D., P. A. Moss, et al. (1996). “Phenotypic analysis of antigen-specific T lymphocytes.” Science 274(5284): 94-96.
Bachmann, M. F., B. Ecabert, et al. (1999). “Influenza virus: a novel method to assess viral and neutralizing antibody titers in vitro.” J Immunol Methods 225(1-2): 105-111.
Banerjee, I., Y. Yamauchi, et al. (2013). “High-Content Analysis of Sequential Events during the Early Phase of Influenza A Virus Infection.” PLoS One 8(7): e68450.
Bui, M., G. Whittaker, et al. (1996). “Effect of M1 protein and low pH on nuclear transport of influenza virus ribonucleoproteins.” J Virol 70(12): 8391-8401.
Bukrinskaya, A. G., N. K. Vorkunova, et al. (1982). “Influenza virus uncoating in infected cells and effect of rimantadine.” J Gen Virol 60(Pt 1): 49-59.
Chou, Y. Y., N. S. Heaton, et al. (2013). “Colocalization of different influenza viral RNA segments in the cytoplasm before viral budding as shown by single-molecule sensitivity FISH analysis.” PLoS Pathog 9(5): e1003358.
Fauci, A. S. (2006). “Emerging and re-emerging infectious diseases: influenza as a prototype of the host-pathogen balancing act.” Cell 124(4): 665-670.
Firestone, A. J., J. S. Weinger, et al. (2012). “Small-molecule inhibitors of the AAA+ ATPase motor cytoplasmic dynein.” Nature 484(7392): 125-129.
Greber, U. F., I. Singh, et al. (1994). “Mechanisms of virus uncoating.” Trends Microbiol 2(2): 52-56.
Hao, R., P. Nanduri, et al. (2013). “Proteasomes activate aggresome disassembly and clearance by producing unanchored ubiquitin chains.” Mol Cell 51(6): 819-828.
Horvath, P., T. Wild, et al. (2011). “Machine learning improves the precision and robustness of high-content screens: using nonlinear multiparametric methods to analyze screening results.” J Biomol Screen 16(9): 1059-1067.
Hubbert, C., A. Guardiola, et al. (2002). “HDAC6 is a microtubule-associated deacetylase.” Nature 417(6887): 455-458.
Husain, M. and K. S. Harrod (2009). “Influenza A virus-induced caspase-3 cleaves the histone deacetylase 6 in infected epithelial cells.” FEBS Lett 583(15): 2517-2520.
Kawaguchi, Y., J. J. Kovacs, et al. (2003). “The deacetylase HDAC6 regulates aggresome formation and cell viability in response to misfolded protein stress.” Cell 115(6): 727-738.
Kemler, I., G. Whittaker, et al. (1994). “Nuclear import of microinjected influenza virus ribonucleoproteins.” Virology 202(2): 1028-1033.
Lamb, R. A., S. L. Zebedee, et al. (1985). “Influenza virus M2 protein is an integral membrane protein expressed on the infected-cell surface.” Cell 40(3): 627-633.
Martin, K. and A. Helenius (1991). “Nuclear transport of influenza virus ribonucleoproteins: the viral matrix protein (M1) promotes export and inhibits import.” Cell 67(1): 117-130.
Martin, K. and A. Helenius (1991). “Transport of incoming influenza virus nucleocapsids into the nucleus.” J Virol 65(1): 232-244.
Matlin, K. S., H. Reggio, et al. (1981). “Infectious entry pathway of influenza virus in a canine kidney cell line.” J Cell Biol 91(3 Pt 1): 601-613.
Nevalainen, M., M. Nissinen, et al. (2010). “Influenza virus infection in multinucleated skeletal myofibers.” Exp Cell Res 316(11): 1784-1794.
Noda, T. and Y. Kawaoka (2012). “Packaging of influenza virus genome: robustness of selection.” Proc Natl Acad Sci USA 109(23): 8797-8798.
Nusinzon, I. and C. M. Horvath (2006). “Positive and negative regulation of the innate antiviral response and beta interferon gene expression by deacetylation.” Mol Cell Biol 26(8): 3106-3113.
O'Neill, R. E., R. Jaskunas, et al. (1995). “Nuclear import of influenza virus RNA can be mediated by viral nucleoprotein and transport factors required for protein import.” J Biol Chem 270(39): 22701-22704.
Ouyang, H., Y. O. Ali, et al. (2012). “Protein aggregates are recruited to aggresome by histone deacetylase 6 via unanchored ubiquitin C termini.” J Biol Chem 287(4): 2317-2327.
Paschal, B. M., E. L. Holzbaur, et al. (1993). “Characterization of a 50-kDa polypeptide in cytoplasmic dynein preparations reveals a complex with p150GLUED and a novel actin.” J Biol Chem 268(20): 15318-15323.
Pinto, L. H., L. J. Holsinger, et al. (1992). “Influenza virus M2 protein has ion channel activity.” Cell 69(3): 517-528.
Sakai, T., M. Ohuchi, et al. (2006). “Dual wavelength imaging allows analysis of membrane fusion of influenza virus inside cells.” J Virol 80(4): 2013-2018.
Serrador, J. M., J. R. Cabrero, et al. (2004). “HDAC6 deacetylase activity links the tubulin cytoskeleton with immune synapse organization.” Immunity 20(4): 417-428.
Shakespear, M. R., M. A. Halili, et al. (2011). “Histone deacetylases as regulators of inflammation and immunity.” Trends Immunol 32(7): 335-343.
Shaw, M. L., K. L. Stone, et al. (2008). “Cellular proteins in influenza virus particles.” PLoS Pathog 4(6): e1000085.
Singh, I. and A. Helenius (1992). Nucleocapsid uncoating during entry of enveloped animal RNA viruses into cells. Seminars in Virology, HARCOURT BRACE JOVANOVICH.
Soderholm, J. F., S. L. Bird, et al. (2011). “Importazole, a small molecule inhibitor of the transport receptor importin-beta.” ACS Chem Biol 6(7): 700-708.
Strunze, S., M. F. Engelke, et al. (2011). “Kinesin-1-mediated capsid disassembly and disruption of the nuclear pore complex promote virus infection.” Cell Host Microbe 10(3): 210-223.
Suomalainen, M. and U. F. Greber (2013). “Uncoating of non-enveloped viruses.” Curr Opin Virol 3(1): 27-33.
True, O. and P. Matthias (2012). “Interplay between histone deacetylases and autophagy—from cancer therapy to neurodegeneration.” Immunol Cell Biol 90(1): 78-84.
White, J., K. Matlin, et al. (1981). “Cell fusion by Semliki Forest, influenza, and vesicular stomatitis viruses.” J Cell Biol 89(3): 674-679.
Wurzer, W. J., O. Planz, et al. (2003). “Caspase 3 activation is essential for efficient influenza virus propagation.” EMBO J 22(11): 2717-2728.
Yamauchi, Y., H. Boukari, et al. (2011). “Histone deacetylase 8 is required for centrosome cohesion and influenza A virus entry.” PLoS Pathog 7(10): e1002316.
Yang, X. J. and E. Seto (2008). “The Rpd3/Hda1 family of lysine deacetylases: from bacteria and yeast to mice and men.” Nat Rev Mol Cell Biol 9(3): 206-218.
Zhang, Y., B. Gilquin, et al. (2006). “Two catalytic domains are required for protein deacetylation.” J Biol Chem 281(5): 2401-2404.
Zhang, Y., N. Li, et al. (2003). “HDAC-6 interacts with and deacetylates tubulin and microtubules in vivo.” EMBO J 22(5): 1168-1179.
Zhirnov, O. P. (1990). “Solubilization of matrix protein M1/M from virions occurs at different pH for orthomyxo- and paramyxoviruses.” Virology 176(1): 274-279.

1. A method for treating an influenza virus infection in a subject in need thereof comprising administering a therapeutically effective amount of a modulator of the ubiquitin-binding property of HDAC6 to said subject.

2. The method of claim 1 wherein the modulator inhibits the ubiquitin-binding property of HDAC6.

3. The method of claim 1, wherein the influenza virus is influenza A.

4. The method of claim 1, wherein the modulator of HDAC6 decreases or silences the expression of HDAC6.

5. The method of claim 4 wherein the modulator is a siRNA.

6. The method of claim 1, wherein the modulator of HDAC6 is an antibody specifically binding to HDAC6.

7. The method of claim 7 wherein the antibody is a single-domain antibody.

8. An antibody binding to HDAC6 and decreasing or blocking its ubiquitin-binding properties for use in treating an influenza virus infection.

9. The antibody of claim 8, wherein said antibody is a single-domain antibody.


SEQUENCE LISTING (6 sequences)

SEQ ID NO: 1 - DNA, 21 bp, Homo sapiens
caccgtcaacgtggcatggaa

SEQ ID NO: 2 - DNA, 21 bp, Homo sapiens
cacttcgaagcgaaatattaa

SEQ ID NO: 3 - DNA, 21 bp, Homo sapiens
cacggttaatgaagtctgcta

SEQ ID NO: 4 - DNA, 19 bp, Homo sapiens
aacgagattgccaagcata

SEQ ID NO: 5 - DNA, 25 bp, Homo sapiens
gagtctgagcgtgcttccaggaata

SEQ ID NO: 6 - DNA, 25 bp, Homo sapiens
ggagaccattctccacgccttcaaa